(19) United States
(12) Patent Application Publication
(10) Pub. No.: US 2025/0228530 A1
(43) Pub. Date: Jul. 17, 2025

(54) MEDICAL APPARATUS

(71) Applicant: FUJIFILM Corporation, Tokyo (JP)

(72) Inventor: Yutaro KAWAKAMI, Tokyo (JP)

(73) Assignee: FUJIFILM Corporation, Tokyo (JP)

(21) Appl. No.: 19/006,025

(22) Filed: Dec. 30, 2024

(30) Foreign Application Priority Data
Jan. 12, 2024 (JP) 2024-002919

(51) Int. Cl. A61B 8/00; G16H 40/40

(52) U.S. Cl. CPC A61B 8/54; G16H 40/40; A61B 8/461

(57) ABSTRACT

A microcontroller controls a display of a medical apparatus and stores log data representing an event occurred in the medical apparatus in a non-volatile storage device in response to an instruction from a main processor. In a case where the processor cannot start the operating system for some reason, the microcontroller detects the input of the user to the photodiode and the microphone by the power from the power supply, and recognizes the log data output instruction. Then, the microcontroller outputs the log data in the non-volatile storage device as a sound signal from, for example, the speaker.

### CROSS REFERENCE TO RELATED APPLICATION

This application claims priority to Japanese Patent Application No. 2024-002919 filed on Jan. 12, 2024 which is incorporated herein by reference in its entirety including the specification, claims, drawings, and abstract.

### BACKGROUND OF THE DISCLOSURE

### 1. Field of the Disclosure

The present disclosure relates to a medical apparatus, and particularly to a mechanism for collecting data from a medical apparatus.

### 2. Description of the Related Art

Many medical apparatuses such as an ultrasound diagnostic apparatus incorporate a computer. In a case where a power switch of the medical apparatus is turned on, an operating system stored in a boot disk of a computer incorporated in the medical apparatus is started, and various types of software necessary for the operation of the medical apparatus are executed under the control of the operating system.

In addition, in many medical apparatuses, log data on an operation executed by the medical apparatus, log data on an event occurring in the medical apparatus, or the like is recorded in, for example, a built-in non-volatile storage device, for example, for cause analysis of a failure. In a case where a failure occurs in the medical apparatus, a maintenance engineer dispatched by a manufacturer or the like reads out log data from a storage device and analyzes a cause of the failure.

For example, JP2018-11622A shows a mechanism for recording information on a shock applied to an operation panel of a medical apparatus in a memory so that the information can be referred to later.

JP2016-151874A discloses a system that reads out data stored in a storage device in a device to the outside even in a case where an application for data communication via a universal serial bus (USB) of the device cannot be started. In this system, a data output terminal is connected to a USB terminal of the device, and power is supplied from the data acquisition terminal to the device. Then, the data acquisition terminal communicates with the device in accordance with a communication standard of a layer lower than the application layer, and reads out data from a storage device in the device.

### SUMMARY OF THE DISCLOSURE

However, in a case where the main processor or the boot disk of the medical apparatus is out of order, the operating system of the medical apparatus does not start even in a case where the power switch is turned on. In a case where the operating system is not started, various user interfaces and data input and output devices provided in the medical apparatus do not operate, and thus data such as log data stored in the storage device cannot be read out.

An object of the present disclosure is to provide a mechanism that can read out stored data from a medical apparatus in which an operating system is not started.

A medical apparatus disclosed in the present specification is a medical apparatus controlled by an operating system, comprising: a storage device that stores data; one or more sensors that detect a predetermined physical quantity; an output device that is capable of outputting a sound signal or a light signal; a controller that determines whether or not there is an instruction to read out data from a user based on a detection result of the one or more sensors, reads out the data from the storage device in a case where it is determined that there is the instruction to read out the data, and executes control for causing the output device to output the sound signal or the light signal representing the read-out data; and a power supply unit that is capable of supplying power to the storage device, the one or more sensors, the output device, and the controller even in a case where the operating system is not started.

In a case where the operating system is not started, the medical apparatus can instruct the controller to output data from the storage device by the user performing a predetermined input to the one or more sensors. Then, the controller reads out the data from the storage device in response to the instruction, outputs the data as a sound signal or a light signal from the output device, and the user receives the sound signal or the light signal on his/her terminal to acquire the data in the terminal.

Here, the one or more sensors may be a plurality of sensors that detect different types of physical quantities as the sensor, and the controller may determine that the instruction for reading out is given in a case where each of the plurality of sensors output predetermined detection results.

In this configuration, in a case where the user does not perform an appropriate input to a plurality of sensors that detect different types of physical quantities, the data cannot be instructed to be output to the controller. Therefore, there is less risk that the controller is operated by the user unintentionally touching an individual sensor or the like.

The plurality of sensors may be provided at separate positions in a housing of the medical apparatus that are unlikely to be touched simultaneously by the user in a case where the user holds or operates the medical apparatus.

In this manner, the risk that the user unintentionally operates the controller is further reduced.

In addition, the controller may cause, in a case where the data is read out from the storage device and is prepared to be output from the output device in response to the instruction to read out, the output device to output a predetermined sound signal or light signal indicating that the preparation is completed, and the output device to output a sound signal or a light signal representing the data read out from the storage device after the predetermined sound signal or light signal is output.

In this configuration, since the output device is used for two different purposes, an increase in the scale of the apparatus is suppressed.

The medical apparatus may further comprise a second storage device that stores software executed by the controller, and a reception device that receives a sound signal or a light signal representing update data for updating the software, in which the controller determines whether or not there is an instruction to update the software from a user based on a detection result of the one or more sensors, receives an update data transmitted as the sound signal or the light signal by an external device via the reception device in a case where it is determined that the instruction to update the software is given, and update the software in the second storage device using the received update data.

In this configuration, the software for the controller can be updated without going through the operating system.

The one or more sensors may detect the sound signal or the light signal and may also serve as the reception device.

In this configuration, since the one or more sensors are used for two different purposes, an increase in the scale of the apparatus is suppressed.

In the present disclosure, data stored in a medical apparatus whose operating system has not started can be read out.

### BRIEF DESCRIPTION OF THE DRAWINGS

FIG. 1 is a diagram showing an appearance of an example of a medical apparatus.

FIG. 2 is a diagram showing an example of a hardware configuration of a medical apparatus.

FIG. 3 is a diagram illustrating a state transition of a microcontroller of a medical apparatus.

FIG. 4 is a diagram showing another example of a hardware configuration of the medical apparatus.

### DESCRIPTION OF THE EMBODIMENTS

Hereinafter, an example of forms for carrying out the present disclosure (hereinafter, referred to as an embodiment) will be described with reference to the drawings.

A concept of the medical apparatus 100 described below includes, for example, an examination apparatus such as an ultrasound diagnostic apparatus or an X-ray CT apparatus, or a treatment apparatus such as an ultrasound treatment apparatus or a radiation treatment apparatus.

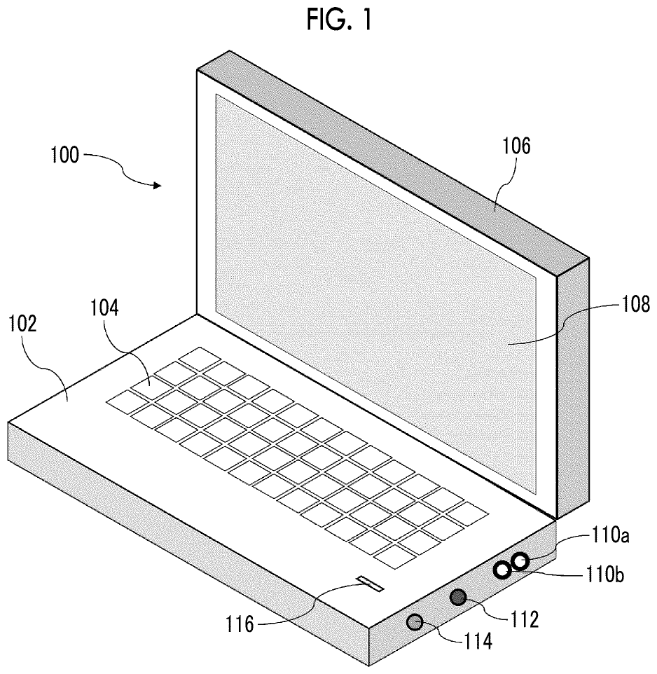

FIG. 1 schematically shows an appearance of an exemplary medical apparatus 100. The exemplary medical apparatus 100 has a structure in which a top part 106 provided with a display 108 is attached to a body part 102 provided with a keyboard to be openable and closable, as in a notebook personal computer.

An input buttons such as a keyboard 104 and an indicator 116 are provided on an upper surface of a housing of the body part 102. The indicator 116 is used for presenting information to the user in the control of the present embodiment. For example, the indicator 116 is configured by a light emitting diode (LED). In this case, predetermined information is presented depending on whether or not the indicator 116 emits light and a pattern of blinking.

In addition, two photodiodes 110a and 110b, a microphone 112, and a speaker 114 are provided on a side surface of the housing of the body part 102. The photodiodes 110a and 110b and the microphone 112 are used to receive an input from the user in the control of the present embodiment. In addition, the speaker 114 is used for presenting information to the user in the control of the present embodiment.

The photodiodes 110a and 110b and the microphone 112 are examples of sensors that detect a predetermined physical quantity. The photodiodes 110a and 110b detect the intensity of light as a physical quantity. In addition, the microphone 112 detects the strength of the sound as a physical quantity. In addition, the indicator 116 and the speaker 114 are examples of output devices capable of outputting a light signal or a sound signal.

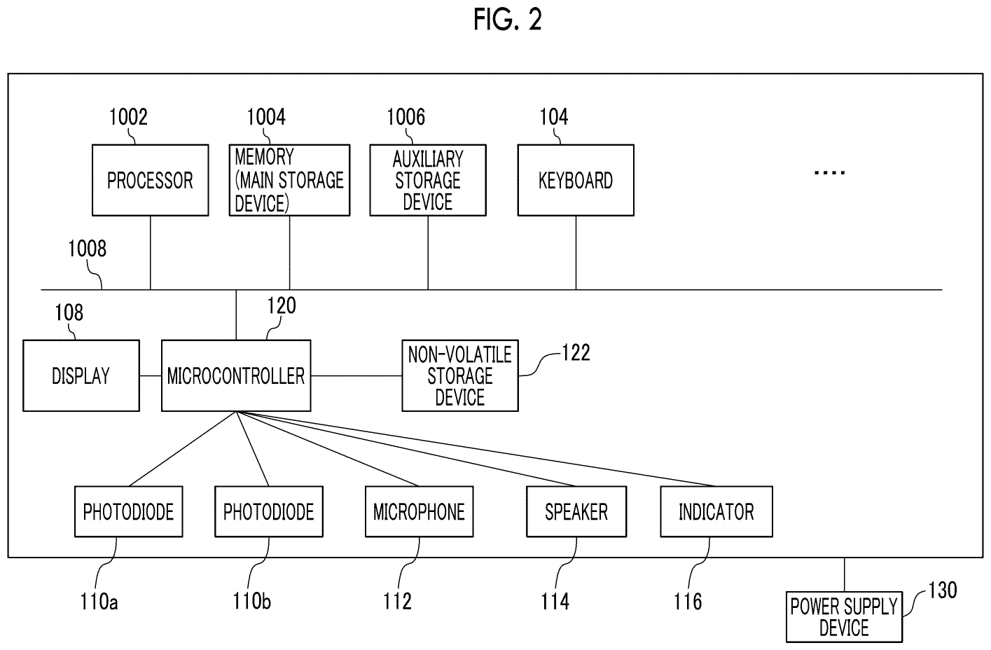

FIG. 2 illustrates a hardware configuration of the medical apparatus 100. In this example, the medical apparatus 100 has a circuit configuration in which a processor 1002, a memory (main storage device) 1004, an auxiliary storage device 1006, a keyboard 104, a microcontroller 120, and the like are connected via, for example, a data transmission path such as a bus 1008.

The processor 1002 is a calculation device that is responsible for main information processing of the medical apparatus 100, and is, for example, a central processing unit (CPU). The memory 1004 is a high-speed main storage device that provides a work area in a case where the processor 1002 executes a program, and is configured as, for example, a random access memory (RAM). The auxiliary storage device 1006 is a large-capacity storage device that stores a program executed by the processor 1002 and various types of data. For example, programs such as firmware, an operating system, and various application software of the medical apparatus 100 are stored in the auxiliary storage device 1006. The auxiliary storage device 1006 is configured by, for example, a non-volatile storage device such as a flash memory, a solid state drive (SSD), or a hard disk drive (HDD). The program, such as the operating system, stored in the auxiliary storage device 1006 is executed by the processor 1002, and thus the medical apparatus 100 exhibits its function.

The microcontroller 120 is an auxiliary processor, and is, for example, for controlling hardware of the medical apparatus 100. In FIG. 2, as an example, the microcontroller 120 is a processor that controls the display 108 of the medical apparatus 100. In a case where the display 108 is, for example, a touch panel display, the microcontroller 120 executes processing such as display on the touch panel display and detection of a touch input.

In the present embodiment, the control processing of the present embodiment is executed by the microcontroller 120 in a case where the operating system of the medical apparatus 100 is not started. It should be noted that the configuration in which the control processing of the present embodiment is carried out by the microcontroller 120 for the display 108 is merely an example. Another microcontroller mounted on the medical apparatus 100 may execute the control processing.

The non-volatile storage device 122 stores firmware of the microcontroller 120. The non-volatile storage device 122 is configured by, for example, a non-volatile storage device such as an SSD, a flash memory, or an HDD. In addition, log data of the medical apparatus 100 is written in the non-volatile storage device 122. That is, the operating system executed by the processor 1002 generates log data indicating various events (for example, occurrence of an error) that occur in the medical apparatus 100, and stores the log data in the non-volatile storage device 122 via the microcontroller 120. That is, in this embodiment, the non-volatile storage device 122 serves as a storage device that stores the log data read out from the medical apparatus 100 to the outside (for example, a terminal of a maintenance engineer) and a second storage device that stores software (for example, firmware) executed by the microcontroller 120.

In addition, the microcontroller 120 controls the photodiodes 110a and 110b, the microphone 112, the speaker 114, and the indicator 116.

The power supply 130 supplies power to the processor 1002 to the bus 1008, and the keyboard 104 to the indicator 116 shown in FIG. 2. In particular, the power supply 130 can supply power to at least the microcontroller 120, the non-volatile storage device 122, the photodiodes 110a and 110B, the microphone 112, the speaker 114, and the indicator 116 even in a case where the processor 1002 cannot start the operating system.

In a case where the processor 1002 cannot start the operating system, the cause of the failure to start the operating system may be clarified in a case where the log data stored in the non-volatile storage device 122 can be read out. However, since the operating system is not started, the log data cannot be viewed or read out to the outside through the user interface provided by the operating system. In the related art, in such a case, it is common that a maintenance engineer of a vendor of the medical apparatus 100 takes the medical apparatus 100 back to a factory of the vendor and investigates a cause of a failure of the medical apparatus 100 in the factory. In addition, there is also a case where the maintenance engineer reads out the log data from the medical apparatus 100 via a wired connection using a dedicated jig in the office on the user side of the medical apparatus 100. However, even in this case, complicated work such as opening the housing of the medical apparatus 100 is required.

On the other hand, in the present embodiment, in a case where the operating system is not started, the user (for example, the maintenance engineer) instructs the microcontroller 120 to output the log data via the photodiodes 110a and 110b, the microphone 112, and the like.

For example, an operation of tapping the microphone 112 with another fingertip a predetermined number of times or more in a state in which the photodiode 110b (hereinafter, also referred to as a photodiode B) is covered with a finger or the like is an operation of giving an instruction to output the log data. In this case, the microcontroller 120 understands that the output of the log data is instructed in a case where the level of the signal from the microphone 112 is equal to or higher than the second threshold value a predetermined number of times or more in a state where the level of the signal from the photodiode B is equal to or lower than the first threshold value. The first threshold value and the second threshold value are predetermined threshold values. It should be noted that the operation for the log data output instruction exemplified here is merely an example.

In a case where this instruction is recognized, the microcontroller 120 outputs, for example, a sound signal representing the log data read out from the non-volatile storage device 122 from the speaker 114. The user receives the sound signal by a microphone of a terminal such as a smartphone and decodes the sound signal to obtain the log data. The voice communication between the microcontroller 120 and the terminal is performed by, for example, ultrasonic communication. The voice communication can be performed using an existing voice communication protocol such as Chirp or Super sonic communication (SSC). By using the ultrasonic communication, data communication can be performed at a speed of, for example, about 2 kbps.

In addition, the microcontroller 120 may receive an update of the firmware in the non-volatile storage device 122 by the user's operation on the photodiodes 110a and 110b, the microphone 112, and the like. Although the following is merely an example, in the following, it is assumed that an operation of tapping the microphone 112 with another fingertip a predetermined number of times or more in a state in which the photodiode 110a (hereinafter, also referred to as a photodiode A) is covered with a finger or the like is an operation of giving an instruction to update the firmware. The microcontroller 120 that recognizes the instruction detects a sound signal representing the update data output from the speaker of the terminal of the user by the microphone 112, decodes the signal to acquire the data, and updates the firmware in the non-volatile storage device 122 by using the data.

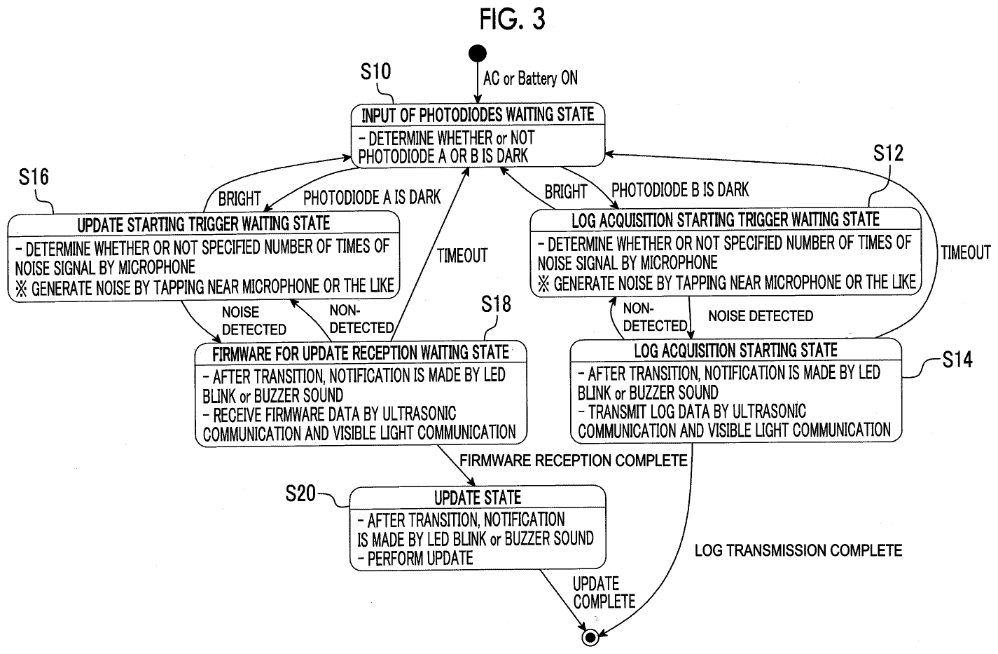

FIG. 3 shows a state transition diagram of the microcontroller 120 in this example. The operation of the microcontroller 120 will be described in more detail with reference to this figure.

The microcontroller 120 transitions to the specific mode, for example, in a case where the processor 1002 cannot start the operating system. In this specific mode, the microcontroller 120 performs the state transition shown in FIG. 3.

During the execution of the specific mode, power is supplied from the power supply 130 to at least the microcontroller 120, the non-volatile storage device 122, the photodiodes 110a and 110b, the microphone 112, the speaker 114, and the indicator 116.

In this mode, the microcontroller 120 first enters a state S10. The state S10 is a state in which the input signals from the photodiodes A and B are being waited for. In the state S10, the microcontroller 120 determines whether or not the photodiodes A and B are dark, that is, whether or not the level of the input signal from each of the photodiodes A and B is greater than the first threshold value. The first threshold value is set to a value sufficiently lower than the level of the signal output by the photodiodes A and B in a normal indoor environment. For example, in a case where the user covers the photodiode A with a finger, the level of the signal output by the photodiode A is equal to or lower than the first threshold value.

Here, in a case where it is determined that the photodiode B is dark, that is, the level of the input signal from the photodiode B is equal to or less than the first threshold value, the microcontroller 120 transitions to the state S12. A state S12 is a state in which an operation that triggers the acquisition of the log data is being waited for. In the state S12, the microcontroller 120 determines whether or not the noise is picked up by the microphone 112 a predetermined number of times or whether or not the number of times of reception of the signal having a level equal to or higher than the second threshold value from the microphone 112 is equal to or more than a predetermined number of times.

In a case where the user wants to acquire the log data, for example, the user covers the photodiode B with a finger of the right hand and repeatedly taps the microphone 112 with another finger of the right hand a predetermined number of times or more in that state. Then, the microphone 112 transmits a signal indicating the noise in a case of being tapped to the microcontroller 120 several times.

In a case where the microphone 112 receives the signal having a level equal to or higher than the second threshold value a predetermined number of times or more, the microcontroller 120 transitions to a state S14.

In a case where the level of the input signal from the photodiode B is larger than the first threshold value before the microcontroller 120 transitions from the state S12 to the state S14, the microcontroller 120 returns to the state S10. In addition, although not shown, in a case where the microphone 112 does not detect noise a predetermined number of times or more from a point in time when the transition is made from the state S10 to the state S12 until a predetermined time elapses, the microcontroller 120 may be returned to the state S10.

A state S14 is a log acquisition start state. In a case where the transition to this state is made, the microcontroller 120 notifies the user that the preparation of the log data transmission is completed, for example, by causing the indicator 116 to blink or causing the speaker 114 to output, for example, a buzzer sound. Thereafter, the microcontroller 120 starts voice communication with the terminal of the user in accordance with a predetermined voice communication protocol. Then, the microcontroller 120 encodes the log data read out from the non-volatile storage device 122 into a sound signal according to the protocol, and outputs the sound signal from the speaker 114. The speaker 114 is used for both output of a notification sound indicating that preparation of transmission of the log data is completed and output of a sound signal representing the log data.

The user starts an application that implements the voice communication protocol on his/her terminal and brings the terminal close to the speaker 114 of the medical apparatus 100. The application starts the voice communication with the microcontroller 120 of the medical apparatus 100 according to the protocol and obtains the log data by decoding the sound signal picked up by the microphone of the terminal according to the protocol.

In a case where the transmission of the log data is completed, the microcontroller 120 returns to the state S10.

In a case where the microphone 112 does not detect the noise a predetermined number of times or more from a point in time when the transition is made from the state S12 to the state S14 until a predetermined time elapses, the microcontroller 120 may be returned to the state S10.

The flow of the processing of outputting the log data to the outside by the microcontroller 120 has been described above. Next, a flow of processing of updating the firmware of the microcontroller 120 will be described.

In a case where it is determined that the photodiode A is dark in the state S10, that is, the level of the input signal from the photodiode A is equal to or less than the first threshold value, the microcontroller 120 transitions to the state S16. A state S16 is a state in which an operation that is a trigger for starting the firmware update is being waited for. In the state S16, the microcontroller 120 determines whether or not the microphone 112 has picked up noise a predetermined number of times or more.

In a case where the user wants to update the firmware, the user covers the photodiode A with a certain finger and repeatedly taps the microphone 112 with another finger a predetermined number of times or more in that state. In a case where the microphone 112 receives the signal having a level equal to or higher than the second threshold value a predetermined number of times or more, the microcontroller 120 transitions to a state S18.

In a case where the level of the input signal from the photodiode A is larger than the first threshold value before the microcontroller 120 transitions from the state S16 to the state S18, the microcontroller 120 returns to the state S10. In addition, in a case where the microphone 112 does not detect the noise a predetermined number of times or more from a point in time when the transition is made from the state S16 to the state S18 until a predetermined length of time elapses, the microcontroller 120 may be returned to the state S10.

A state S18 is a state in which the update data is being waited to be received. In a case of transitioning to this state, the microcontroller 120 notifies the user that the update data can be received, for example, by causing the indicator 116 to blink or outputting a specific sound to the speaker 114. A blinking pattern of the indicator 116 or an output sound of the speaker 114 in this case may be different from the blinking pattern or the type of the buzzer sound in the state S14.

Thereafter, the microcontroller 120 starts voice communication with the terminal of the user in accordance with a predetermined voice communication protocol. The voice communication application of the terminal of the user encodes the update data into a sound signal according to a protocol and outputs the sound signal. The microcontroller 120 restores the update data by decoding the sound signal picked up by the microphone 112 according to the protocol. In this example, the microphone 112 is used for both the detection of the tap operation from the user in the state S16 and the reception of the sound signal representing the update data in the state S18.

In a case where the restoration of the update data is completed, the microcontroller 120 transitions to a state S20. A state S20 is a state in which the update of the firmware is being executed. In a case where the transition to this state is made, the microcontroller 120 notifies that the update is started by causing the indicator 116 to blink or causing the speaker 114 to output a specific sound. In addition, the microcontroller 120 updates the firmware in the non- volatile storage device 122 by using the restored update data. In a case where the update is completed, the microcontroller 120 returns to the state S10.

In the example described above, the voice communication protocol is used for the communication between the microcontroller 120 and the terminal of the user in the specific mode. However, this is merely an example, and a communication method using various other media can be used.

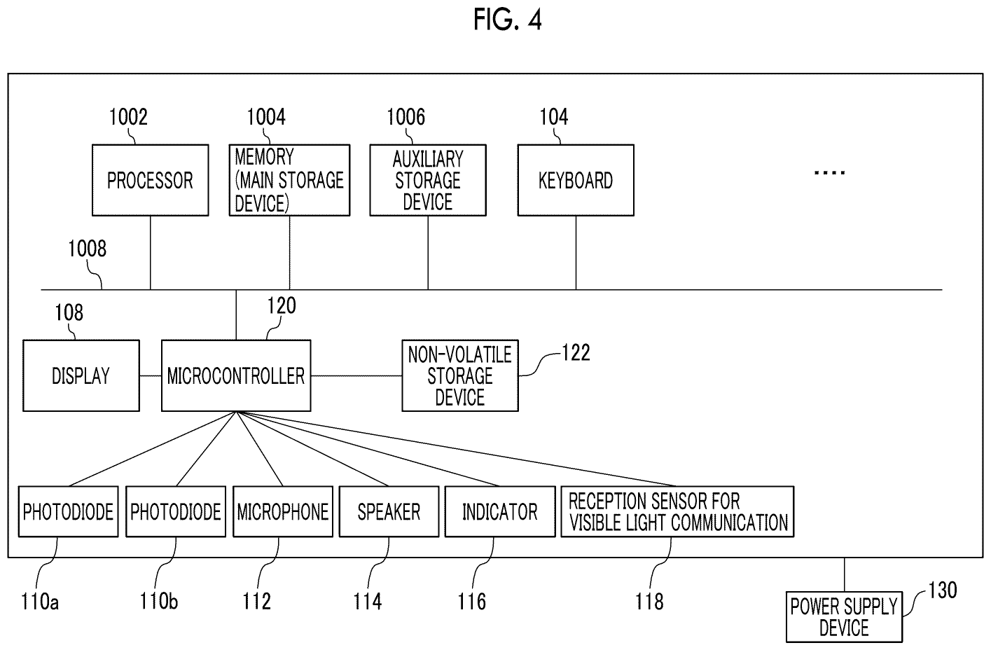

For example, visible light communication may be used. In the example shown in FIG. 4, the medical apparatus 100 includes a reception sensor 118 for visible light communication. In this example, a program that implements the visible light communication protocol is installed in both the microcontroller 120 and the terminal of the user.

In addition, in this example, in a case where the microcontroller 120 transmits the log data to the terminal of the user in the state S14 in the state transition of FIG. 3, the visible light communication is used. That is, the microcontroller 120 transmits the log data to the outside by controlling the luminescence intensity of the indicator 116 in accordance with the log data. The user captures the indicator 116 with the camera of the terminal in a state in which the application for visible light communication of the terminal is started. The application decodes the change in the temporal light intensity of the indicator 116 appearing in the captured moving image to restore the log data.

In addition, in this example, in a case where the update data is transmitted from the terminal of the user to the microcontroller 120 in the state S18 in the state transition of FIG. 3, the visible light communication is used. In this case, the user brings a light emitting device (for example, a lighting lamp for imaging or a display) incorporated in the terminal close to the reception sensor 118 for visible light communication and instructs the application to start transmission of the update data in this state. In response to this instruction, the application transmits the data to the outside by changing the light emission of the light emitting device in a temporal manner according to the update data. The microcontroller 120 of the medical apparatus 100 restores the update data by decoding the signal detected by the reception sensor 118 for visible light communication in accordance with the visible light communication protocol, and updates the firmware using the data.

In the above-described embodiment, a complex operation of tapping the microphone 112 in a state of covering the photodiode 110a or 110b is required as a trigger for starting the log data output, the update, or the like. By using the composite operation as a trigger in this way, it is possible to avoid the malfunction of the microcontroller 120 in a case where the user accidentally touches the photodiodes 110a and 110b or the microphone 112. Here, the photodiode 110a (or 110b) and the microphone 112 are provided at positions slightly away from the side surface of the body part 102. Therefore, in a case where the user operates or holds the medical apparatus 100, it is unlikely that the photodiode 110a (or 110b) and the microphone 112 are simultaneously touched by mistake.

In addition, in order to prevent malfunction, another sensor such as a Hall element may be provided. For example, a hall element is provided in the medical apparatus 100 instead of the photodiode 110a or 110b. The user brings the magnet carried with the user close to the Hall element instead of covering the photodiode 110b with the finger. Accordingly, the microcontroller 120 transitions from the state S10 to the state S12 in the state transition of FIG. 3. In a case of the photodiode 110a or 110b, it is likely that the user unintentionally covers the photodiode 110a or 110b, but in a case of the Hall element, it is necessary to prepare a magnet, so it is unlikely that the Hall element is unintentionally operated.

In addition, in the above-described embodiment, the photodiodes 110a and 110b and the microphone 112 are used as sensors for the microcontroller 120 to receive an operation from the user, but this is merely an example. For example, a vibration pad that detects vibration may be used instead of the microphone 112. In a case where the user taps the vibration pad, a signal indicating the vibration generated by the tapping is transmitted from the vibration pad to the microcontroller 120.

In the above example, the control processing of the present embodiment is executed by the microcontroller 120 that controls the specific hardware in the medical apparatus 100, but this is merely an example. A dedicated microcontroller that performs the control processing of the present embodiment may be provided in the medical apparatus 100.

The embodiments and the modification examples of the present disclosure have been described above. These embodiments and modification examples are merely examples for description. Various modifications and improvements can be made within the scope of the present disclosure.

What is claimed is:

1. A medical apparatus controlled by an operating system, comprising:
a storage device that stores data;
one or more sensors that detect a predetermined physical quantity;
an output device that is capable of outputting a sound signal or a light signal;
a controller that determines whether or not there is an instruction to read out data from a user based on a detection result of the one or more sensors, reads out the data from the storage device in a case where it is determined that there is the instruction to read out the data, and executes control for causing the output device to output the sound signal or the light signal representing the read-out data; and
a power supply unit that is capable of supplying power to the storage device, the one or more sensors, the output device, and the controller even in a case where the operating system is not started.

2. The medical apparatus according to claim 1,
wherein the one or more sensors are a plurality of sensors that detect different types of physical quantities, and the controller determines that the instruction for reading out is given in a case where each of the plurality of sensors output predetermined detection results.

3. The medical apparatus according to claim 2,
wherein the plurality of sensors are provided at separate positions in a housing of the medical apparatus that are unlikely to be touched simultaneously by the user in a case where the user holds or operates the medical apparatus.

4. The medical apparatus according to claim 1,
wherein the controller causes, in a case where the data is read out from the storage device and is prepared to be output from the output device in response to the instruction to read out, the output device to output a predetermined sound signal or light signal indicating that the preparation is completed, and the output device to output a sound signal or a light signal representing the data read out from the storage device after the predetermined sound signal or light signal is output.

5. The medical apparatus according to claim 1, further comprising:
a second storage device that stores software executed by the controller; and
a reception device that receives a sound signal or a light signal representing update data for updating the software,
wherein the controller
determines whether or not there is an instruction to update the software from a user based on a detection result of the one or more sensors,
receives an update data transmitted as the sound signal or the light signal by an external device via the reception device in a case where it is determined that the instruction to update the software is given, and
updates the software in the second storage device using the received update data.

6. The medical apparatus according to claim 5,
wherein the one or more sensors detect the sound signal or the light signal and also serves as the reception device.
